(19) United States
(12) Patent Application Publication
(10) Pub. No.: US 2007/0282343 A1
(43) Pub. Date: Dec. 6, 2007

(54) Bone Cutter

(75) Inventors: Atsushi Hashimoto, Tokyo (JP); Jyun Kimura, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: NIHON UNIVERSITY, TOKYO (JP)

(21) Appl. No.: 10/582,581

(22) PCT Filed: Nov. 29, 2004
(86) PCT No.: PCT/JP04/17683

§ 371 Date: Apr. 2, 2007

(30) Foreign Application Priority Data
Dec. 15, 2003 (JP) 2003-416589

(51) Int. Cl. A61B 17/00

(52) U.S. Cl. 606/79

(57) ABSTRACT

A bone cutter for cutting a bone by pulling a wire wrapped around the bone is provided. With the bone cutter, it is easy to fix a cutting wire at a part to be cut of a bone and, when the bone is cut, the bone neither vibrates nor moves. The bone cutter includes a cylindrical main section (2); a first supporting wire (W2) an end portion of which is connected to a longitudinal end (5) of the main section (2); a second supporting wire (W3) an end portion of which is connected to the other longitudinal end (6) of the main section (2); a fastener (4) to which the other ends (8) and (9) of the first and the second supporting wires (W2) and (W3), respectively, are fastened; a cutting wire (W1) one end of which is fastened to the fastener (4) and which forms a circularly curved portion (Wr); a pulling section (3) which pulls a free end (13) of the cutting wire (W1) to reduce a diameter of the circularly curved portion; and an adjusting section (25A) which adjusts a pulling force applied to the end portion (6A) of the second supporting wire (W3).

### TECHNICAL FIELD

The present invention relates to a bone cutter for cutting a bone by wrapping a wire around the bone and pulling the wire.

### BACKGROUND ART

It is well known that a saw is used to cut a bone (see, for example, the patent document 1).

The conventional technique, however, has a drawback that a bone being cut by a saw is, by the force applied to the saw, vibrated or moved without being fixed in a position, so that the bone cannot be cut in a stable condition.

Other techniques have also been proposed, including a proximal femur osteotomy device used for artificial hip joint placement (see the patent document 2, for example), and a high tibial osteotomy apparatus, osteotomy guide apparatus and osteotomy method (see the patent document 3, for example).

These conventional techniques are, however, for osteotomy for a specific part of a body, so that they are not suitable for use in general bone resection.

The inventor of the present invention, while studying ceramics cutting, has found out that ceramics cutting techniques (see the patent document 4, for example) can also be applied to bone cutting. What the present invention proposes is based on what the inventor's study has achieved.

Patent document 1: Japanese Published Unexamined Patent Application No. 1998-137253

Patent document 2: Japanese Published Unexamined Patent Application No. 1993-240964

Patent document 3: Japanese Published Unexamined Patent Application No. 2001-293004

Patent document 4: Japanese Published Unexamined Patent Application No. 2002-308636

### DISCLOSURE OF THE INVENTION

Problems to be Solved by the Invention

The present invention has been made in view of the above problems with the conventional techniques. It is an object of the present invention to provide a bone cutter which enables a cutting wire to be fixed with ease at a part to be cut of a bone and which can cut the bone without causing the bone to vibrate or move.

Means for Solving the Problems

The bone cutter according to the present invention, denoted by numeral 1 in FIG. 1, is for cutting a bone by pulling a wire wrapped around an outer circumference of the bone. The most principal characteristic of the bone cutter is that it includes: a cylindrical main section 2; a first supporting wire W2 an end portion of which is connected to a longitudinal end 5 of the main section 2; a second supporting wire W3 an end portion of which is connected to the other longitudinal end 6 of the main section 2; a fastener 4 to which the other ends 8 and 9 of the first and the second supporting wires W2 and W3, respectively, are fastened; a cutting wire W1 one end of which is fastened to the fastener 4 and which forms a circularly curved portion Wr to cut a bone; a pulling section 3 which pulls a free end 13 of the cutting wire W1 to reduce the diameter of the circularly curved portion Wr; and an adjusting section 25A which adjusts the pulling force applied to a fixture 6A at an end portion of the second supporting wire W3 (claim 1).

The pulling section 3 for pulling the cutting wire W1 and the adjusting section 25A for adjusting the pulling force applied to the second supporting wire W3 may be operated manually, electrically, or hydraulically. When fixing an end of the cutting wire W1 to the fastener 4 and forming the circularly curved portion Wr, a fixture capable of releasing the one end of the cutting wire W1 from the fastener 4 may be made use of.

The pulling section 3 is preferably configured such that the free end 13 of the cutting wire W1 is pulled by a screw jack 21 including a first threaded rod 22 and a first nut 23 installed in the main section 2 (claim 2).

In the above configuration, turning the first nut 23 screwed on the first threaded rod 22 moves the first nut 23 along the first threaded rod 22, thereby allowing the screw jack 21 to operate. Consequently, the free end 13 of the cutting wire W1 is pulled together with the first nut 23 to reduce the diameter of the circularly curved portion Wr.

The pulling section 3 preferably includes a first cylindrical part 32, at an upper end portion 15 of which the free end 13 of the cutting wire W1 is fixed by the fixing section 35 and a lower end portion 16 of which is disposed to be in contact with an upper portion of the first nut 23 (claim 3).

In an embodiment of the present invention, the free end 13 of the cutting wire W1 is fixed to a member disposed above the first cylindrical part 32. In this configuration, turning the first nut 23 screwed on the first threaded rod 22 included in the pulling section 3 moves the first nut 23 along the first threaded rod 22, causing the free end 13 of the cutting wire W1 to be pulled to reduce the diameter of the circularly curved portion Wr.

Preferably, the first cylindrical part 32 has a slit 18 extending in the axial direction thereof and a rotation preventing part 10 fixed, through the slit 18, to the first threaded rod 22 is provided (claim 4).

In this configuration, when the first cylindrical part 32 ascends pulling the cutting wire W1, the first cylindrical part 32 is prevented from rotating, so that the cutting wire W1 is prevented from being twisted. The slit 18 is required to be longer than the stroke traveled by the cutting wire W1 when it is pulled.

Preferably, the pulling section 3 is provided with a fixing section 35 which fixes the circularly curved portion Wr of the cutting wire W1 where the circularly curved portion Wr comes in contact with a bone. The fixing section 35 preferably includes a fixture 35a to which the free end 13 of the cutting wire W1 is fixed, a third threaded rod 26 which supports the fixture 35a, and a third nut 27 which fixes a pulling position of the third threaded rod 26 (claim 5).

In this configuration, the circularly curved portion Wr of the cutting wire W1 is wrapped around accurately at a part to be cut of a bone, and the cutting wire W1 is prevented from being shifted when the bone is cut.

Preferably, the adjusting section 25A that adjusts the pulling force applied to the end portion 6 of the second supporting wire W3 includes a second threaded rod 24 and a second nut 25 installed in the main section 2 (claim 6).

In an embodiment of the present invention, a cylindrical part 34 which moves together with the second nut 25 is installed, and an end portion of the second supporting wire W3 is fixed to an end portion of the cylindrical part 34 using the fixture 6A.

In this configuration, the fastener 4 at the end portion of the cutting wire W1 can be moderately fixed by tightening or loosening the second supporting wire W3, so that the cutting wire W1 can be wrapped around a part to be cut of a bone with ease.

The cutting wire to be used in implementing the present invention is preferably a flexible one having a diameter of 0.2 to 4 mm. It is desired to be made of titan, stainless steel, steel, reinforced plastic, or other material having enough strength to cut a bone. For example, a fiber string may also be used as the cutting wire. The cutting wire may be a solid, nonstranded wire, but using a stranded wire is also effective in implementing the present invention.

Effect of the Invention

The present invention has the following advantageous effects:

(a) An end portion of a circularly curved portion of a cutting wire can be moderately fixed using a fastener to which first and second supporting wires are fastened, so that the circularly curved portion of the cutting wire can be flexibly moved and so that the circularly curved portion of the cutting wire can be wrapped around a part to be cut of a bone with ease.

(b) When a pulling section for pulling the cutting wire is made in a screw jack configuration including a first threaded rod and a first nut, a large pulling force can be obtained to enable smooth pulling operation.

(c) When a cylindrical part is disposed on top of the first nut included in the pulling section for pulling the cutting wire, a free end of the cutting wire can be fixed to an upper portion of the cylindrical part with ease.

(d) When the cylindrical part is provided with a slit which extends in the axial direction of the cylindrical part and to which a slip preventing part is attached, turning the first nut included in the pulling section does not cause the cylindrical part to rotate, so that the cutting wire is not twisted.

(e) When the pulling section is provided with a fixture for fixing the circularly curved portion, in a state of being in contact with a bone, of the cutting wire, the circularly curved portion can be wrapped around accurately at a part to be cut of the bone not to cause the circularly curved portion to be shifted from the cutting position.

(f) When an adjusting section for adjusting the pulling force applied to the second supporting wire is provided, the fastener to which the cutting wire is fastened can be moderately fixed making it easy to wrap the cutting wire around a bone.

### BEST MODE FOR CARRYING OUT THE INVENTION

FIGS. 1 to 5 illustrate an embodiment of a bone cutter according to the present invention. FIG. 6 and Table 1 show results of testing carried out using the bone cutter.

The bone cutter as shown in its entirety in FIG. 1 has, as its main components, a main section 2 to which an end of a cutting wire W1, and first and second supporting wires W2 and W3 are connected, and a pulling section 3 for pulling a free end, that is, the other end of the cutting wire W1.

The main section 2 is, as shown in FIG. 2, comprised of a cylindrical first main part 2A, a half cylindrical main part 2B fixed to the left side, as viewed in FIG. 2, of the main part 2A, and a cylindrical third main part 2C fixed to the right side, as viewed in FIG. 2, of the main part 2A.

The cylindrical first main part 2A has an inner hole 2Ai, and a cylindrical projection 2D which projects, forming a T shape together with the first main part 2A, from an approximate longitudinal center of the first main part 2A. The projection 2D has an inner hole 2Di reaching the inner hole 2Ai.

An edge 7 where the inner hole 2Ai and the inner hole 2Di meet is rounded providing a smooth surface over which the cutting wire W1 can slide smoothly.

The second main part 2B, as shown in FIGS. 4 and 5, has a cut-away surface 14 which, excluding both end portions, extend in parallel with the axis of the second main part 2B and which forms part of a space where the first and the second supporting wires W2 and W3, and a fastener 4 are operated.

The second main part 2B also has a slit-like groove, not illustrated in the figures, which extends in parallel with the axis of the second main part 2B and whose depth is perpendicular to the cut-away surface 14. The groove extending in an inner hole provided along the axis of the second main part 2B reaches an inner hole 2Ai of the first main part 2A.

The free end side of a circularly curved portion Wr of the cutting wire W1 is arranged to be operable in the groove.

A fourth threaded rod 28 is attached to an end of the second main part 2B. It is clamped to the second main part 2B by a fourth nut 29 which fixes an end portion of the first supporting wire W2.

The third main part 2C includes, as shown together with the first main part 2A in FIG. 2, a second threaded rod 24 connected with a screw 2As to the right end portion, as viewed in FIG. 2, of the main part 2A, a second nut 25 screwed on the second threaded rod 24, a second cylindrical part 34 being in contact with the second nut 25 and covering the second threaded rod 24, and a disk 34a being in contact with the cylindrical part 34 and being able to function as a washer.

The second threaded rod 24 and the second nut 25 make up an adjusting section 25A for adjusting the pulling force applied to the second supporting wire W3.

Rotating the second nut 25 screwed on the second threaded rod 24 causes the second cylindrical part 34 to move together with the second nut 25. Thus, a jacking function for moving the second cylindrical part 34 is provided.

The second cylindrical part 34 is attached with a slit for rotation prevention and a rotation preventing part similar to that provided for a first cylindrical part 32 which is being described later.

The second threaded rod 24 is provided with an inner hole 24i reaching the inner hole 2Ai of the first main part 2A in such a configuration that the second supporting wire W3 runs through the inner holes.

The main section 2 configured as described above is provided with the first and the second supporting wires W2 and W3 as described below.

As shown in FIGS. 4 and 5, two free ends of the first supporting wire W2 looped back inside the half cylindrical main part 2B are fixed with the fourth nut 29 screwed on the fourth threaded rod 28. An end 8 formed by looping back of the first supporting wire W2 is fastened to a hole 4a formed in the fastener 4.

Two free ends of the second supporting wire W3 looped back inside the half cylindrical main part 2B are fixed to a fixture 6A externally attached, as shown in FIG. 2, to the second cylindrical part 34. An end 9 formed by looping back of the second supporting wire W3 is fastened to the hole 4a formed in the fastener 4.

In addition to the hole 4a to which the ends 8 and 9 formed by looping back of the first and the second supporting wires W2 and W3, respectively, are fastened as described above, the fastener 4 also has a hole 4b to which, as being described later, one end of the circularly curved portion Wr of the cutting wire W1 is fixed.

As shown in FIGS. 2 and 3, the pulling section 3 for pulling the cutting wire W1 has, as its main components, a first threaded rod 22 screwed to the projection 2D of the main part 2A, a first nut 23 screwed on the first threaded rod 22 and attached with a wrench 23A, the first cylindrical part 32 being in contact with the first nut 23 and covering the first threaded rod 22, and a fixing section 35 which fixes the circularly curved portion Wr of the cutting wire W1 where the circularly curved portion Wr comes in contact with a bone.

The first threaded rod 22 has an inner hole 22i. A third threaded rod 26 having an inner hole 26i is inserted in the inner hole 22i.

The first threaded rod 22 and the first nut 23 make up a screw jack 21. The jacking function of the screw jack 21 is materialized by rotating the first nut 23 using the wrench 23A. The operation generates a large force to cause the first nut 23 and the third threaded rod 26 to be moved together.

The first cylindrical part 32 covering the first threaded rod 22 is disposed on top of the first nut 23. An annular disk 32a being able to function as a washer is disposed on top of the first cylindrical part 32. A third nut 27 screwed on the third threaded rod 26 is disposed on top of the disk 32a so that it can determine the height position of the third threaded rod 26.

The first cylindrical part 32 has a slit 18 extending in the axial direction thereof. A sliding piece 11 and a fastening screw 12 make up a rotation preventing part 10 which is fixed, by the fastening screw 12, to the first threaded rod 22 through the slit 18. The rotation preventing part 10 prevents the first cylindrical part 32 from being rotated even when the first nut 23 is rotated, so that the cutting wire W1 is prevented from being twisted.

A fixture 35a to which a free end 13 of the cutting wire W1 is fixed is disposed on top of a hexagonal head 26a of the third threaded rod 26. The third threaded rod 26, the third nut 27, and the fixture 35a make up the fixing section 35 which fixes the circularly curved portion Wr of the cutting wire W1 where the circularly curved portion Wr comes in contact with a bone.

The route along which the cutting wire W1 runs through the pulling section 3 and the main section 2 configured as described above will be described below.

The cutting wire W1 preferably has a diameter of up to 3 mm.

As shown in FIG. 4, the first and the second supporting wires W2 and W3 are fastened to the hole 4a provided in the fastener 4, and an end portion We of the cutting wire W1 is fastened to plural fastening holes 4b provided in an end portion of the fastener 4.

The cutting wire W1 extending from the fastener 4 toward the fourth nut 29 forms the circularly curved portion Wr, and further extends toward the main part 2A. It then enters the inner hole 2Ai shown in FIG. 2 and extends into the pulling section 3 shown in FIG. 3 via the rounded sliding portion 7.

The cutting wire W1 further runs through the inner hole 22i of the first threaded rod 22 and the inner hole 26i of the third threaded rod 26. Finally, at its free end 13, the cutting wire W1 is fixed to the fixture 35a.

How the bone cutter 1 configured as described above operates is described in the following with reference to FIG. 1.

First, the second nut 25 included in the adjusting section 25A for adjusting the pulling force applied to the second supporting wire W3 is turned until the fastener 4 is moderately fixed. At this time, it may also be possible to adjust the first supporting wire W2 using the fourth nut 29.

Next, the diameter of the circularly curved portion Wr of the cutting wire W1 is determined based on the part to be cut of a bone and the diameter of the part to be cut. The diameter of the circularly curved portion Wr is adjustable by turning the first nut 23 included in the pulling section 3 for pulling the free end 13 of the cutting wire W1. It can also be adjusted by turning the third nut 27 included in the fixing section 35 disposed above the pulling section 3. Furthermore, the diameter of the circularly curved portion Wr may also be adjusted using a fixture which can be arbitrarily clamped in place of the fixture 35a provided at the free end 13 of the cutting wire W1.

Now, the cutting wire W1 has been adjusted to be ready for use.

Next, the circularly curved portion Wr of the cutting wire W1 is wrapped around an outer circumference of the bone. Since the circularly curved portion Wr can be moved in any direction within an appropriate range, it can be wrapped around the bone with ease.

The circularly curved portion Wr is then tightly fixed to the bone not to be shifted when its diameter is reduced. This is done by turning the third nut 27 included in the fixing section 35.

Now, preparation for cutting the bone is complete.

Subsequently, the bone is cut by turning, by the use of the wrench 23A, the first nut 23 included in the pulling section 21 that is used to pull the free end 13 of the cutting wire W1 to reduce the diameter of the circularly curved portion Wr.

Pig bone cutting tests carried out using the above-description function will be described in the following with reference to FIG. 6 and Table 1. Table 1 shows results of the test.

TABLE 1

| Test | | Bone cut | Cutting load |
|---|---|---|---|
| No. | Bone from | at | kN |
| 1 | Hind foot of | A | 1.40 |
| | little pig | | |
| 2 | Fore foot of | A | 2.90 |
| | adult pig | | |
| 3 | Fore foot of | A | 2.25 |
| | adult pig | | |
| 4 | Hind foot of | A | 1.50 |
| | adult pig | | |
| 5 | Hind foot of | B | 4.55 |
| | adult pig | | |

FIG. 6 shows a typical shape of the bones used in the tests and parts where the bones were cut. According to FIG. 6, a foot bone 40 was cut at an end part along a chain line denoted by A, or it was cut at a middle part along a chain line denoted by B.

Table 1 shows information relevant to the above bone cutting tests. The wire used in the tests had a diameter of 3 mm.

Table 1 covers test numbers 1 to 5. Namely, it gives, on each of the test numbers 1 to 5, information such as “bone from” indicating what part of what pig the tested bone was from, “bone cut at” indicating what part of the bone under test was cut, and “cutting load kN” indicating the cutting load applied in the test.

In test number 2, for example, a bone from a fore foot of an adult pig was cut at its part A (along a chain line denoted by A in FIG. 6) by applying a cutting load, i.e., a tensile load of 2.90 kN to the cutting wire W1.

In all the tests, numbers 1 to 5, the bone under test having a dual structure which includes a stiff external surface and a soft inner part was clearly cut as expected.

The conclusions obtained from the tests are as follows.

(1) The cutting load is affected by the age of the pig from which the bone to be cut is obtained. Namely, the cutting load required to cut a bone of an aged pig is larger than that required to cut a bone of a young pig.

(2) The required cutting load differs depending on whether the bone to be cut is from a fore foot or from a hind foot of a pig. Namely, the cutting load required to cut a bone of a fore foot of a pig is larger than that required to cut a bone of a hind foot of a pig.

(3) Even though different parts of a bone can be cut, the cutting load required varies with the part to be cut.

In view of the test results obtained, the bone cutter 1 according to the present invention has been confirmed to be effective.

### BRIEF DESCRIPTION OF THE DRAWINGS

FIG. 1 is a block diagram showing an embodiment of a bone cutter according to the present invention.

FIG. 2 is a sectional side view of a main section shown in FIG. 1.

FIG. 3 is a sectional side view of a pulling section, shown in FIG. 1, for pulling a cutting wire.

FIG. 4 is a side view of a cutting wire, a first supporting wire, and a second supporting wire shown in FIG. 1.

FIG. 5 is a top view corresponding to FIG. 4.

FIG. 6 is a diagram showing a typical shape of bones used in testing a bone cutter according to the present invention and parts at which the bones were cut.

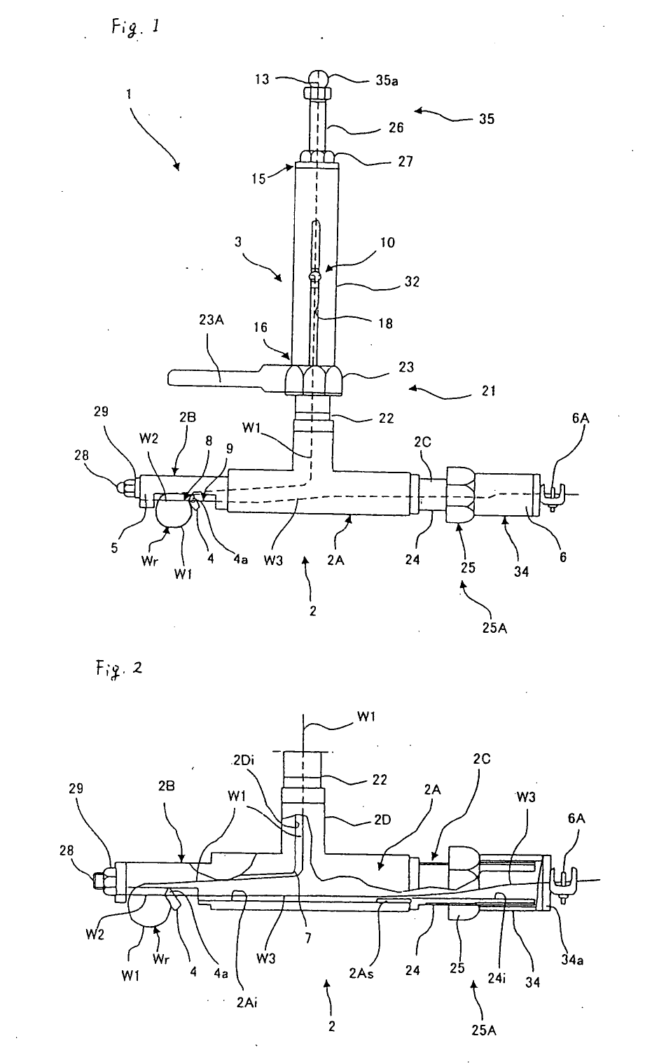

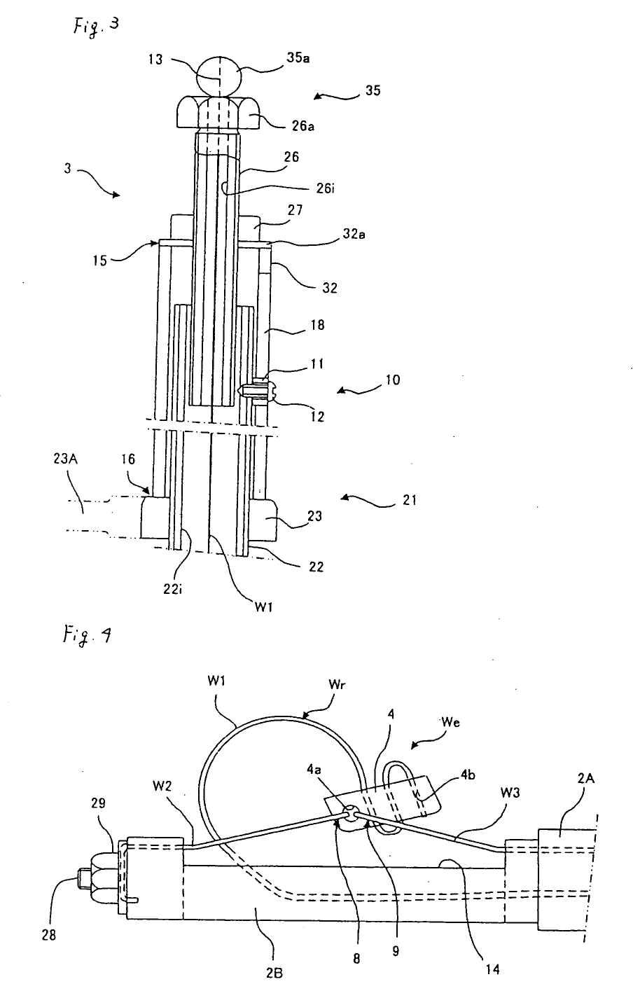

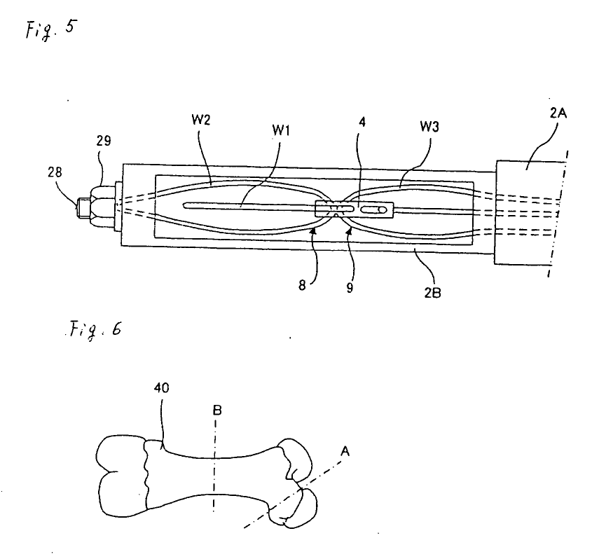

### DESCRIPTION OF REFERENCE NUMERALS AND SYMBOLS

W1 cutting wire
We fixed end
Wr circularly curved portion (diameter of circularly curved portion)
W2 first supporting wire
W3 second supporting wire
1 cutter
2 main section
2A first main part
2B second main part
2C third main part
2D projection
2Ai, 2Di, 22i, 24i, 26i inner hole
2As screw
3 pulling section
4 fastener
4a hole
4b fastening hole
5, 6 end portion
6A fixture for fixing end portion of second supporting wire
7 rounded sliding portion (edge)
8, 9 other end
10 rotation preventing part
11 sliding piece
12 screw
13 free end of cutting wire
14 cut-away surface
15 upper end portion
16 lower end portion
18 slit
21 screw jack
22 first threaded rod
23 first nut
23A wrench
24 second threaded rod
25 second nut
25A adjusting section
26 third threaded rod
26a hexagonal head
27 third nut
28 fourth threaded rod
29 fourth nut
32 first cylindrical part
32a, 34a disk
34 second cylindrical part
35 fixing section
35a fixture for fixing free end portion of-cutting wire
40 foot bone

1. A bone cutter for cutting a bone by pulling a wire wrapped around the bone, comprising;
a cylindrical main section, a first supporting wire an end portion of which is connected to a longitudinal end of the main section,
a second supporting wire an end portion of which is connected to the other longitudinal end of the main section,
a fastener to which the other ends of both the first and the second supporting wires are fastened,
a cutting wire one end of which is fastened to the fastener and which forms a circularly curved portion,
a pulling section which pulls a free end of the cutting wire to reduce a diameter of the circularly curved portion, and
an adjusting section which adjusts a pulling force applied to the end portion of the second supporting wire.

2. The bone cutter according to claim 1, wherein the pulling section is configured such that the free end of the cutting wire is pulled by a screw jack including a first threaded rod and a first nut installed in the main section.

3. The bone cutter according to claim 2, wherein the pulling section includes a cylindrical part at an upper end portion of which the free end of the cutting wire is fixed and a lower end portion of which is disposed to be in contact with an upper portion of the first nut.

4. The bone cutter according to claim 3, wherein the cylindrical part has a slit extending in an axial direction thereof and wherein a rotation preventing part fixed, through the slit, to the first threaded rod is provided.

5. The bone cutter according to one of claims 1 to 4, wherein the pulling section is provided with a fixing section which fixes the circularly curved portion of the cutting wire where the circularly curved portion comes in contact with a bone, the fixing section including a fixture to which the free end of the cutting wire is fixed, a third threaded rod which supports the fixture, and a third nut which fixes a position of the third threaded rod.

6. The bone cutter according to one of claims 1 to 4, wherein the adjusting section that adjusts the pulling force applied to the end portion of the second supporting wire includes a second threaded rod and a second nut installed in the main section.
